(19) United States
(12) Patent Application Publication
(10) Pub. No.: US 2017/0333062 A1
(43) Pub. Date: Nov. 23, 2017

(54) Tongue cleaner with a mouth freshener disinfecting strip

(71) Applicants: Paritosh Patel, Pismo Beach, CA (US); Vinay Jivan, Dublin, CA (US); Anita Jivan, Dublin, CA (US)

(72) Inventors: Paritosh Patel, Pismo Beach, CA (US); Vinay Jivan, Dublin, CA (US); Anita Jivan, Dublin, CA (US)

(21) Appl. No.: 15/159,811

(22) Filed: May 20, 2016

(51) Int. Cl. A61B 17/24; A61C 19/06

(52) U.S. Cl. CPC A61B 17/244; A61C 19/06

(57) ABSTRACT

A self-contained tongue cleaner having a strip/band of material to provide a method of disinfecting and freshening the tongue as it is stroked against the tongue. The improvement comprises of a disinfecting/mouth freshener strip or gel located on the head in close proximity to the tongue scraper edge. The strip material will easily dissolve when in contact with the tongue and hence deliver the disinfecting/freshening fluid directly to the tongue.

### FIELD OF THE INVENTION

This invention relates to a system that improves good oral hygiene by cleaning and disinfecting the surface of your tongue. In particular, this invention relates to a disposable tongue cleaner that not only cleans the surface of the tongue but also kills bacteria and freshens the tongue, unlike traditional tongue cleaners that only remove the debris buildup from the tongue.

### BACKGROUND OF THE INVENTION

A toothbrush is used to clean teeth, but only a few people use the conventional brush to brush their tongue to remove plaque and debris from their tongue. Conventional toothbrushes with bristles are often uncomfortable to use on the tongue and hence many people don't use it to clean their tongue. In Asian countries people have been using tongue scrapers for many centuries. These scrapers are typically a U shape curved metal, made from stainless steel or copper and are used to clean the tongues.

These scrapers help remove plaque and debris from the tongue but do a poor job in killing the bacteria that resides on the tongue. Hence, there is an unmet need that toothbrushes and tongue scrapers need to overcome in order to enhance oral hygiene.

### SUMMARY OF THE INVENTION

This invention centers on a unique design of a tongue scraper that can deliver cleaning, disinfecting, or other solutions directly to the surface of the tongue allowing for optimum removal of plaque and debris from your tongue.

The present disclosure provides an oral care implementation that includes a head that forms the scraper which channels the plaque and in close proximity to the scraper's edge is a parallel strip of material that can easily dissolve when in contact with the tongue. The strip of material could be a gel or a solid that would quickly dissolve when in contact with the tongue; hence the solution would be directly applied to the contact area.

### BRIEF DESCRIPTION OF DRAWINGS

FIG. 1: Side view of a tongue cleaner in accordance with and/or use with the present invention.

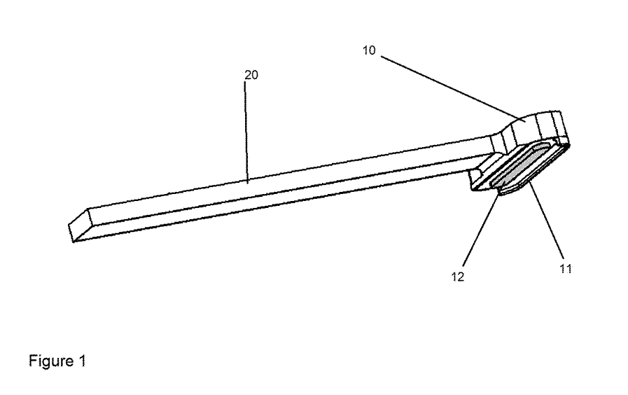

FIG. 2: Shows a close up of the head and the parts associated, the edge acts as the scraper and a strip of disinfecting or freshening material being in close proximity to the scraper edge.

FIG. 3: Showing how the strip pattern can be changed to allow for different fluids to be applied to the tongue at the same time.

FIG. 4: Showing the tongue cleaner having two edges and the strip being in the middle.

FIG. 5: A tongue cleaner having a placeholder/cavity for one to place disinfecting/cleaning gel or strip at the time of use.

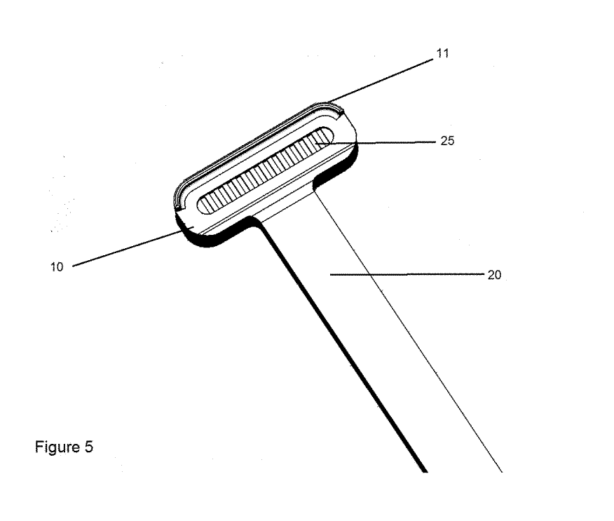

### REFERENCE NUMERALS IN DRAWINGS

10 Head part of cleaner
11 Beveled scraper edge
12 Dissolving strip material
14 Strip pattern accommodating different disinfecting or freshening material
15 Secondary beveled scraper edge
20 Handle part of cleaner
25 Placeholder for strip

### DETAILS OF THE INVENTION

The present invention relates to a tongue cleaner designed to aid in a wide variety of oral hygiene. The tongue cleaner has a handle (20) and attached to it a head (10). The low profile head incorporates: a scraper (11) and a strip of disinfection material (12) in close proximity of the scraper. The strip of disinfecting material (12) is located so that it is virtually in constant contact with the tongue during the tongue cleaners scraping strokes. And once the user is done with the device, the device would be thrown away.

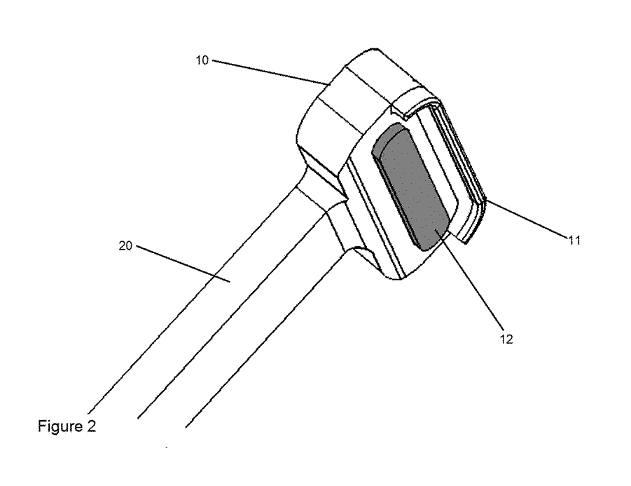

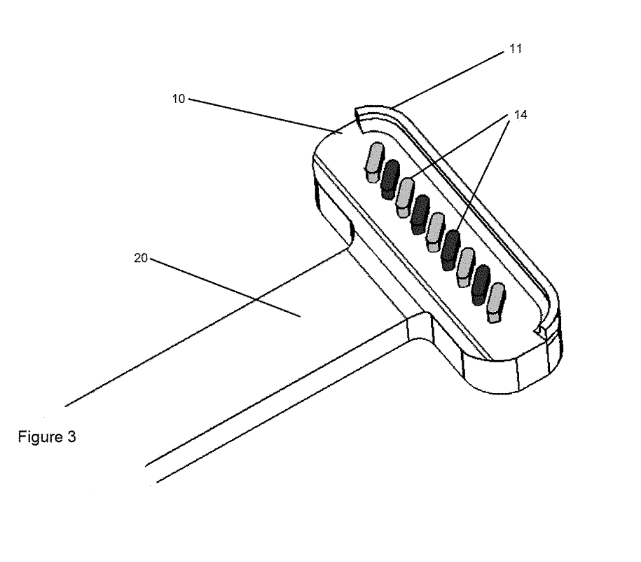

The strip of course could be of different dimension than that shown in FIG. 2 or FIG. 3. It could be made of a series of smaller strips (14) such as FIG. 3 allowing for different disinfecting/cleaning solutions to be applied to the tongue. The strip itself can also be made with different solubility hence can act as a wear indicator, when no strips are seen it is time to dispose the device.

The strip itself could also be made to be two layers of material. For example, the first layer could be a disinfecting gel and the second layer could be a freshening gel. So in this case the first few strokes of the tongue cleaner would remove the debris and disinfect the tongue and the subsequent strokes would apply a freshener to the tongue.

The disinfecting/cleaning strip would normally be part of the tongue cleaner. However, there is also the possibility were the tongue cleaner has a place holder (25) for the strip, and at the time of use the strip is applied (if a gel) or pressed onto the place holder to form the strip.

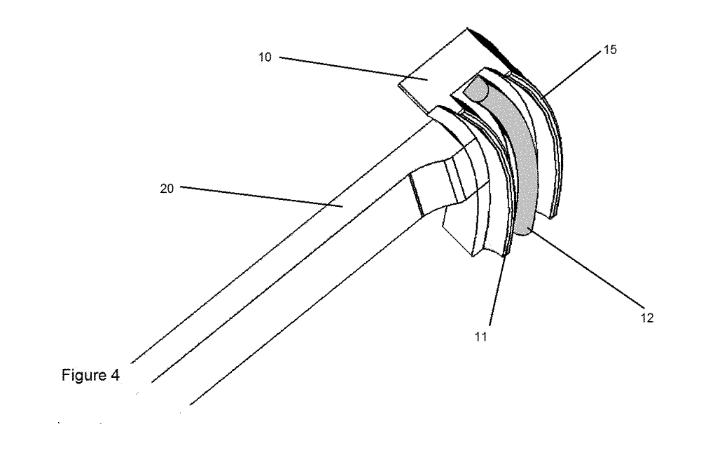

1. A tongue cleaner having at least one scraper edge mounted in a head (10) or frame, the improvement comprising of a soluble strip (12) containing disinfecting/freshening material that is located on the head or frame in close proximity to the edge: The strip of material (12), (14) extending generally parallel to the edge will dissolve as the tongue cleaner is stroked against the tongue, so not only will the buildup debris be removed but the disinfecting/freshening material will also kill the bacteria and freshen the tongue.
Variations to the strip (12) size, shape, strip layers and solubility are within the scope of the accompanying claims as long as the variations don't depart or sacrifice the advantages and principles of the invention.
The strip (12) could also act as a wear indicator, once the strip gone, it would be time to replace the tongue cleaner.
Also as to who attaches the strip, the manufacturer of the tongue cleaner or the end user by applying a gel or by press fitting a strip in a place (25) holder/cavity are all within this claim.
The tongue cleaner head (10) as shown in FIG. 4 could have dual scraper edges (11), (15) with the disinfecting/freshening strip (12) being located in the middle. In this design the first edge (11) would remove the plaque and debris, the strip (12) would then cover the cleaned surface with disinfection/freshening material and the second edge (15) would help spread and remove any excess of the disinfecting/freshening material from the tongue surface. Having such variations of the number of scraper edges and strips pattern variations are within the scope of this claim.
